(19) United States
(12) Patent Application Publication
(10) Pub. No.: US 2008/0107751 A1
(43) Pub. Date: May 8, 2008

(54) Antimicrobial Wax Composition for Wax Therapy

(76) Inventors: Jeff Hudnall, Wolfforth, TX (US); Mindy Malynn Brashears, Lubbock, TX (US)

Correspondence Address: STORM LLP, BANK OF AMERICA PLAZA, 901 MAIN STREET, SUITE 7100, DALLAS, TX, 75202, US

(21) Appl. No.: 10/592,077

(22) PCT Filed: Mar. 10, 2005
(86) PCT No.: PCT/US05/07621

§ 371 Date: Jun. 25, 2007

Related U.S. Application Data
(60) Provisional application No. 60/551,938, filed on Mar. 10, 2004.

(51) Int. Cl. A61K 31/01; A61K 35/12; A61K 36/00; A61K 36/886; A61K 8/92; A61Q 19/00

(52) U.S. Cl. 424/522; 424/725; 424/744; 514/762

(57) ABSTRACT

A wax composition comprises a wax and an antimicrobial agent in an amount effective to inhibit the growth of micro-organisms in the wax in both solid and liquid states. According to the preferred embodiment of the present invention, the wax composition comprises a wax that is solid at room temperature, an anti-microbial agent in an amount effective to inhibit the growth of micro-organisms in the wax in the liquid state, and the balance of the composition is at least one oil that is liquid at room temperature.

### BACKGROUND OF THE INVENTION

1. Technical Field

The present invention relates generally to wax compositions for use in “wax therapy” in which a person's hand, foot, elbow or other appendage is dipped or immersed in molten wax. More particularly, the present invention relates to paraffin wax compositions for use in wax therapy that have improved sanitation and hygiene characteristics.

2. Summary of the Prior Art

It is quite popular at spas and nail salons to treat the hands, feet, and/or other appendages of a patient with “wax therapy,” typically in conjunction with a manicure or pedicure. Such therapy also has application in physical therapy settings, where it is employed to deliver heat therapy for pain relief and management purposes. In wax therapy, the patient's appendage is immersed in a relatively low-temperature (˜120° F.) bath of molten wax. The appendage then is removed from the bath and, after cooling (and sometimes repeated immersions), the wax is peeled off while still soft. According to its proponents, the wax transfers heat to the appendage and also removes dead skin cells, and has smoothing and emollient effects on the skin of the patient.

Typically, the wax used in these baths is conventional paraffin wax, which may or may not have a fragrance, emollient oils, and even coloring added to enhance the pleasure of the wax therapy experience for the patient, and to increase the soothing and moisturizing characteristics of the therapy.

Currently, technology concerning wax therapy centers provides the solid (pre-melt) wax in a conveniently useful form, wherein portions or chunks of the wax may be broken off a larger piece as needed (U.S. Pat. No. 6,190,672 to Anderson) and a “microwavable” wax formulation (U.S. Patent Application Publication 2003/0003118 to Boilen et at.). Also, improvements in the heaters and baths used to melt and contain the wax have been made (U.S. Pat. No. 6,441,388 to Yang et al.).

Because the wax and wax bath may be used with several patients, one after another, there is concern on the part of both patients and therapists about the cleanliness and hygienic properties of the molten wax. Patients may be reluctant to immerse an appendage in a visibly “dirty” bath, and there is always concern about transmission of disease in “reused” or recycled wax, even if it does not appear to be “dirty.” Transmission of a disease or infection from one patient to another from use of a therapeutic wax bath could give rise to liability on the part of the bath operator. U.S. Pat. No. 6,627,072 to Ridden discloses a pumping and filtration system that filters particulate matter from molten wax and return it to the bath for reuse. This apparatus is complex and no doubt costly. Alternatively, the wax can be replaced after every use, which can be expensive and time-consuming.

The applicant and inventor has commissioned studies of the ability of bacteria to grow and survive in molten paraffin of the type used in wax therapy baths. The pathogens tested for growth and survival included Staphylococcus aureus, E. Coli 0157, Salmonella enteritidis and Salmonella typhimurium, Streptococcus mutans, and Streptococcus typhillus, each of which is a fairly typical or representative human pathogen. The study determined that molten wax presents a generally inhospitable environment to these pathogens so that growth of the pathogens was limited. Nevertheless, Streptococcus, Staphylococcus, and E. Coli demonstrated an ability to survive in the molten wax for an hour or more, raising the possibility of transmission of these pathogens from one patient to another.

Accordingly, a need exists for wax compositions that address the perceived and real risk of transmission of disease from the re-use of wax in wax therapy baths.

### SUMMARY OF THE INVENTION

It is a general object of the present invention to provide a wax composition for use in wax therapy baths that is more hygienic than conventional wax compositions.

This and other objects of the present invention are achieved by providing a wax composition comprising a wax and an antimicrobial agent in an amount effective to inhibit the growth of micro-organisms in the wax in both solid and liquid states. According to a preferred embodiment of the present invention, the wax composition comprises a wax that is solid at room temperature, an anti-microbial agent in an amount effective to inhibit the growth of micro-organisms in the wax in both solid and liquid states, and the balance of the composition is at least one oil that is liquid at room temperature.

According to a preferred embodiment of the present invention, the anti-microbial agent is triclosan, which is present in an amount not less than about 0.18-0.2% by weight.

According to a preferred embodiment of the present invention, the oil includes mineral oil.

According to a preferred embodiment of the present invention, the oil includes one or more oil selected from thin group of animal and vegetable oils consisting of jojoba oil, emu oil, and aloe vera oil.

According to a preferred embodiment of the present invention, a fragrance is added.

According to a preferred embodiment of the present invention, a color is added.

According to a preferred embodiment of the present invention, the wax is paraffin wax.

According to a preferred embodiment of the present invention, the wax composition comprises about 95% by weight paraffin wax, about 4.5% by weight mineral oil, about 0.05% by weight of at least one oil selected from the group of oils consisting of jojoba oil, emu oil, and aloe vera oil, and an anti-microbial agent in an amount effective to inhibit the growth of micro-organisms in the wax in both solid and liquid states.

Other objects, features, and advantages of the present invention will become apparent with reference to the detailed description of the invention, which follows.

### DETAILED DESCRIPTION OF THE INVENTION

The wax composition according to the present invention is adapted and intended for use in therapeutic wax baths, whether used in connection with purely cosmetic or aesthetic purposes, such as in a nail salon or spa, or in physical therapy settings, where wax baths are used as a form of heat therapy. In either case, the wax composition according to the present invention addresses perceived and real concerns relating to the growth of microorganisms, especially human pathogens, in the wax used in such baths (and associated potential for disease transmission) while simultaneously avoiding or mitigating the need to filter or treat the wax between uses or to simply replace the wax.

In its simplest form, the wax composition according to the present invention comprises a wax component and an antimicrobial component. The preferred wax is conventional, physio-therapy-grade paraffin wax that is solid at room temperature and has a melting point around 120° F. The preferred antimicrobial agent or component is triclosan, which is a phenolic, broad-spectrum antimicrobial that is generally regarded as safe for use on human skin, and even for ingestion by humans in small amounts. It is available from several sources, including Ciba Specialty Chemicals Corporation, 4090 Premier Drive, High Point, N.C. 27265. Triclosan has been found to inhibit growth of pathogens in wax (as described herein) maintained at a temperature of 52.5° C. or 126.5° F. in concentrations as low as 500 μg/ml. Specifically, triclosan has been found to inhibit growth (slow the growth of bacteria to a rate significantly lower than in the absence of triclosan) and decrease: the time of “survivablity” of Salmonella (both enteritidis and typhimurium strains), E. Coli 0157, and Staphylococcus aurea in wax compositions as described below maintained in a molten state. These bacteria were selected for testing as common human pathogens that are commonly found on the hands. Additionally, these pathogens also model the “survivability” of other pathogens that could be found in the melted wax.

It was found that a concentration as low as 500 μg/ml and up to 1000 μg/ml permitted growth and survival of E. Coli 0157, Staphylococcus, and Streptococcus, but not Salmonella (which apparently is eliminated by heat alone). Accordingly, it was found that a concentration of 2000 μg/ml inhibits the growth of both pathogens. This is the preferred concentration of the triclosan antimicrobial in the molten wax composition according to the present invention. As disclosed in connection with the below examples, this is about 0.18-0.2% by weight of the total composition.

Inasmuch as the wax composition according to the present invention must maintain its character as “wax” (solid at room temperature and molten at elevated temperatures still comfortable for contact with human skin, preferably 120° F. or 49-50° C.) for utility in wax therapy, the primary constituent of any composition according to the present invention will be wax. According to the preferred embodiment of the present invention, the wax is a physio-therapy-grade paraffin wax. Other waxes may be appropriate for the invention, but, due to its relatively low cost, paraffin is preferred. It is believed that the wax must comprise at least 40-45% by weight of the composition in order for the composition to retain its character as “wax” (at least what is thought of by most people as “wax”). It is believed that wax should be at least a majority (more than 50%) of the composition. Still more preferably, as disclosed below in connection with the examples, the weight percentage of wax is much higher, approaching 95% by weight of the total composition.

Because the wax composition according to the present invention is used in contact with human skin, a moisturizing component is preferred. The moisturizing component preferably includes mineral oil (a USP formulation of paraffinic and/or other hydrocarbons) that is liquid at room temperature (1 atmosphere or 101 kPa and about 20° C.). Preferably, mineral oil is present in about 4.5% by weight of the composition.

The wax composition according to the present invention may also include animal and vegetable oils such as emu oil, jojoba oil, aloe vera oil, avocado oil, and the like that are also liquid at room temperature. Such oils typically will be mixed and preferably together are present in about 0.05% by weight of the composition. These oils are selected essentially for emollient and cosmetic purposes and the choice is driven in part by consumer preference.

Additionally, and again as driven by consumer preferences, the composition according to the present invention may contain a coloring or dye and/or a fragrance. Often, the color will be dictated by the fragrance: red or green for apple, yellow or green for citrus or melon, orange for orange, and the like. In some cases, a fragrance- and color-free (“unscented”) composition will be preferred, as in the case of potentially allergic therapy patients. The weight percentage of these components as part of the composition is negligible, typically no more than 0.2%.

Following are examples of wax compositions in accordance with the present invention:

### EXAMPLE 1 (“Apple Orchard”)

| Component | Weight (grams) | Weight percent |
|---|---|---|
| Paraffin Wax | 254016.00 | 94.38682 |
| Mineral Oil USP | 11430.72 | 4.24741 |
| Apple Peel (fragrance) | 2540.16 | 0.94387 |
| Triclosan | 508.03 | 0.18877 |
| Red Dye | 508.03 | 0.18877 |
| Emu Oil | 68.58 | 0.02548 |
| Jojoba Oil | 25.40 | 0.00944 |
| Aloe Vera Oil | 25.40 | 0.00944 |

### EXAMPLE 2 (“Cucumber Melon”)

| Component | Weight (grams) | Weight percent |
|---|---|---|
| Paraffin Wax | 254016.00 | 94.56086 |
| Mineral Oil USP | 11430.72 | 4.25524 |
| Cucumber Melon | 2540.16 | 0.94561 |
| (fragrance) | | |
| Triclosan | 508.03 | 0.1892 |
| Emu Oil | 68.58 | 0.02553 |
| Aloe Vera Oil | 25.40 | 0.00946 |
| Jojoba Oil | 25.40 | 0.00946 |
| Honey Dew (dye) | 6.35 | 0.00024 |
| Teal Dye | 6.35 | 0.00024 |

### EXAMPLE 3 (“Orange Vanilla”)

| Component | Weight (grams) | Weight percent |
|---|---|---|
| Paraffin Wax | 254016.00 | 94.53405 |
| Mineral Oil USP | 11430.72 | 4.25403 |
| Orange Vanilla (fragrance) | 2540.16 | 0.94534 |
| Triclosan | 508.03 | 0.1891 |
| Peach 1850 (dye) | 88.90 | 0.00331 |
| Emu Oil | 68.58 | 0.02552 |
| Aloe Vera Oil | 25.40 | 0.77999 |
| Jojoba Oil | 25.40 | 0.00945 |

### EXAMPLE 4 (“Peach”)

| Component | Weight (grams) | Weight percent |
|---|---|---|
| Paraffin Wax | 254016.00 | 94.56131 |
| Mineral Oil USP | 11430.72 | 4.25526 |
| Ginger Peach (fragrance) | 2540.16 | 0.94561 |
| Triclosan | 508.03 | 0.01891 |
| Emu Oil | 68.58 | 0.02553 |
| Jojoba Oil | 25.40 | 0.00946 |
| Aloe Vera Oil | 25.40 | 0.00946 |
| Peach 1850 (dye) | 11.43 | 0.00043 |

### EXAMPLE 5 (“Waterdance”)

| Component | Weight (grams) | Weight percent |
|---|---|---|
| Paraffin Wax | 254016.00 | 94.56310 |
| Mineral Oil USP | 11430.72 | 4.25534 |
| Waterdance (fragrance) | 2540.16 | 0.94574 |
| Triclosan | 508.03 | 0.2000 |
| Emu Oil | 68.58 | 0.02553 |
| Jojoba Oil | 25.40 | 0.00946 |
| Aloe Vera Oil | 25.40 | 0.00946 |
| Blue (dye) | 6.35 | 0.00236 |

### EXAMPLE 6 (“Unscented”)

| Component | Weight (grams) | Weight percent |
|---|---|---|
| Paraffin Wax | 254016.00 | 95.46813 |
| Mineral Oil USP | 11430.72 | 4.29607 |
| Triclosan | 508.03 | 0.1909 |
| Emu Oil | 68.58 | 0.02577 |
| Aloe Vera Oil | 25.40 | 0.00955 |
| Jojoba Oil | 25.40 | 0.00955 |

All of the examples provide the weights and weight percentages of the components prior to mixing. The non-wax components are mixed together and then mixed with molten wax. The mixture is stirred by hand for 4 to 5 minutes, until the dye (if any) is dispersed in the wax.

The foregoing examples vary primarily in their fragrance and coloration. They are by way of example only and not intended to be by way of limitation. Variation of other components is regarded as within the invention. In all examples, the addition of triclosan in the described and preferred amount reduced the growth rate of human pathogens in the example wax compositions as compared to similar compositions without addition of an antimicrobial.

The present invention provides a wax composition for use in wax therapy baths that addresses the perceived and real hygienic threat posed by reuse of the wax without resorting to expensive and unreliable filtration devices. It also avoids the necessity of discarding the used wax after every treatment, which is both expensive and time-consuming (requiring melting of the “fresh” batch of wax).

The invention has been described with reference to preferred embodiments thereof, but is not thus limited. It is susceptible to variation and modification without departing from the scope and spirit of the invention.

I claim:

1. A wax composition for use in a wax bath, the composition comprising: about 94.5% by weight wax; and
an anti-microbial agent in an amount effective to inhibit tie growth of micro-organisms in the wax in the liquid state.

2. The wax composition according to claim 1, further comprising a fragrance.

3. The wax composition according to claim 1, further comprising mineral oil.

4. The wax composition according to claim 1, further comprising at least one oil selected from the group of animal and vegetable oils consisting of jojoba oil, emu oil, and aloe vera oil.

5. The wax composition according to claim 1, wherein the wax is paraffin wax.

6. The wax composition according to claim 1, farther comprising a color.

7. A wax composition for use in a wax bath, the wax composition comprising:
a wax that is solid at room temperature;
an anti-microbial agent in an amount effective to inhibit the growth of micro-organisms in the wax in the liquid state; and
the balance at least one oil that is liquid at room temperature.

8. The wax composition according to claim 7, wherein the oil includes mineral oil.

9. The wax composition according to claim 7, wherein the oil includes one or more oil selected from the group of animal and vegetable oils consisting of jojoba oil, emu oil, and aloe vera oil.

10. The wax composition according to claim 7, further comprising a fragrance.

11. The wax composition according to claim 7; further comprising a color.

12. The wax composition according to claim 7, wherein the wax is paraffin wax.

13. A wax composition for use in a wax bath, the wax composition comprising:
about 94.5% by weight paraffin wax;
about 4.5% by weight mineral oil;
about 0.05% by weight of at least one oil selected from the group of oils consisting of jojoba oil, emu oil, and aloe vera oil; and
an anti-microbial agent in an amount effective to inhibit the growth of micro-organisms in the wax in the liquid state.

14. The wax composition according to claim 13, further comprising a fragrance.

15. The wax composition according to claim 13, further comprising a color.
